(19) United States
(12) Patent Application Publication
(10) Pub. No.: US 2026/0207445 A1
(43) Pub. Date: Jul. 23, 2026

(54) IMPROVEMENTS IN OR RELATED TO ORGANIC COMPOUNDS

(71) Applicant: GIVAUDAN SA, VERNIER (CH)

(72) Inventors: Antoine CHARBONNIER, Argenteuil (FR); Ly Huong VU, Argenteuil (FR)

(21) Appl. No.: 19/133,546

(22) PCT Filed: Nov. 28, 2023
(86) PCT No.: PCT/EP2023/083273

§ 371 Date: May 28, 2025

(30) Foreign Application Priority Data
Dec. 2, 2022 (GB) 2218130.9

(51) Int. Cl. A61K 8/11; A61K 8/66; A61Q 13/00; A61Q 19/00

(52) U.S. Cl. CPC A61K 8/11; A61K 8/66; A61Q 13/00; A61Q 19/00; A61K 2800/412; A61K 2800/95

(57) ABSTRACT

The present invention provides a microcapsule composition comprising at least one core-shell microcapsule, wherein the at least one core-shell microcapsule comprises a hydrophobic core and a shell surrounding the core, wherein the shell comprises a polymer or oligomer generated in situ by an enzyme-catalysed process.

The present invention relates to a microcapsule composition comprising at least one core-shell microcapsule, to a method for preparing such a microcapsule composition, as well as to the use of such a microcapsule composition to enhance the performance of a benefit agent in a consumer product.

It is known to incorporate encapsulated benefit agents in consumer products, such as household care, personal care and fabric care products. Benefit agents include for example fragrances, cosmetic agents, food ingredients, nutraceuticals, drugs and substrate enhancers.

Encapsulated benefit agents are known in the art. They may be formed by a process of coating small solid particles or liquid droplets in a thin film of shell material. Although virtually any coating material, conceptually at least, is a candidate capsule shell material, in practice for commercial and regulatory reasons, to-date, there are relatively few materials that have been used in commercial products. Capsule shell material selection is determined by a number of factors including final application, cost, availability, processing ease, and inherent barrier properties. Defining an optimal shell material for a given application can be complex since many interacting parameters determine success of a given capsule shell material.

Microcapsules that are particularly suitable for delivery of benefit agents are core-shell microcapsules, wherein the core usually comprises the benefit agent and the shell is impervious or at least partially impervious to the benefit agent. Generally, these microcapsules are employed in aqueous media and the encapsulated benefit agents are hydrophobic. A broad selection of shell materials can be used, provided the shell material is impervious or at least partially impervious to the encapsulated benefit agent.

Benefit agents are encapsulated for a variety of reasons. Microcapsules can isolate and protect such materials from external suspending media, such as consumer product bases, in which they may be incompatible or unstable. They are also used to assist in the deposition of benefit agents onto substrates, such as skin or hair, or also fabrics or hard household surfaces in case of perfume ingredients. They can also act as a means of controlling the spatio-temporal release of a benefit agent.

A wide variety of encapsulating media as well as benefit agents suitable for the preparation of encapsulated compositions has been proposed in the prior art. Such encapsulating media include synthetic resins made from polyamides, polyureas, polyurethanes, polyacrylates, melamine-derived resins, or mixtures thereof. Encapsulated benefit agent compositions are typically prepared in the form of aqueous slurries.

Consumers are increasingly concerned about using materials obtained from non-renewable sources, such as synthetic petrochemicals, as well as about the processes for manufacturing the consumer products. The “clean label” concept is one of the biggest trends of the decade. The term itself has many definitions including sustainable, naturally sourced or bio-based and biodegradable ingredients as well as minimal processing and impact on the environment. Nevertheless, it is generally difficult to use natural materials or materials derived from nature to satisfy the requirements for suitable encapsulation compositions. Bio-based and biodegradable ingredients for customer formulations must provide a unique combination of performance and sustainability, so consumers feel confident in the safety and efficacy of these ingredients.

Nature-derived polymers such as proteins and polysaccharides are attractive chemical building blocks to encapsulate agents and have been explored in the recent years by the industry. Many of these materials are also biodegradable. However, there is a continuous need to provide new biodegradable materials for benefit agent encapsulation.

Generation of macromolecular (oligomeric or polymeric) materials using enzymes as catalysts has been receiving increased interest (Nikulin et al, Molecules 2021, 26, 2750), however no application of such enzymatically-generated oligo- or polymers in encapsulation of benefit agents has been reported.

### SUMMARY OF THE INVENTION

In a first aspect, the invention provides a microcapsule composition comprising at least one core-shell microcapsule, wherein the at least one core-shell microcapsule comprises a hydrophobic core and a shell surrounding the core, wherein the shell comprises a polymer or oligomer generated in situ by an enzyme-catalysed oligomerization/polymerization of a substrate.

In a second aspect, a method for preparing the microcapsule composition as described herein is provided.

In a further aspect, it is provided the use of a microcapsule composition as described herein to enhance the performance of a benefit agent in a consumer product.

The invention further provides a consumer product comprising a microcapsule composition as described herein.

### BRIEF DESCRIPTION OF THE DRAWINGS

FIG. 1a shows an example of reaction kinetics of an esterase-catalysed formation of oligo- and/or polypeptides.

FIG. 1b shows an example of a comparison between the ¹H NMR (H₂O) spectra of (1bi) monomer L-alanine ethyl ester and (1bii) a reaction product polyalanine.

FIG. 2a shows an example of overlayed IR spectra of monomer tyrosine methyl ester and reaction product polytyrosine.

FIG. 2b shows an example of a comparison between the ¹H NMR (H₂O) spectra of (2bi) monomer L-tyrosine methyl ester and (2bii) reaction product polytyrosine.

FIG. 3 shows an example of a microscopic image of a microcapsule composition obtained by a protease-catalysed process employing an alanine ethyl ester substrate, encapsulating Mygliol.

FIGS. 4 to 7 show microscopic images of microcapsule compositions obtained by a protease-catalysed process employing tyrosine methyl ester and N-benzoyl-N-tyrosine ethyl ester (BTEE) substrates, encapsulating various fragrances.

FIG. 8 illustrates a microscopic image of a microcapsule composition obtained by an oxido-reductase-catalysed process employing tyrosine methyl ester and N-benzoyl-N-tyrosine ethyl ester (BTEE) substrates, encapsulating a fragrance.

FIGS. 9a and 10a show microscopic images of microcapsule compositions obtained by a lipase-catalysed process employing a pentadecanolide substrate, encapsulating a fragrance.

FIGS. 9b and 10b show SEM (scanning electron microscopy) images of microcapsule compositions obtained by a lipase-catalysed process employing a pentadecanolide substrate, encapsulating a fragrance.

### DEFINITIONS

The term “benefit agent” refers to any substance which, when added to a product, may improve the perception of this product by a consumer or may enhance the action of this product in an application. Examples of benefit agents include perfume/fragrance ingredients, flavor ingredients, cosmetic ingredients, bioactive agents (such as bactericides, insect repellents and pheromones), substrate enhancers (such as silicones and brighteners), enzymes (such as lipases and proteases), dyes, pigments and nutraceuticals.

The term “microcapsules” refers to capsules of sizes ranging from 0.1 μm to 500 μm.

The term “bio-based” relates to the origin of a material and refers to materials intentionally made from substances derived from living (or once-living) organisms, as opposed to petroleum-derived materials. The definition includes both natural materials, such as naturally-extracted proteins and polysaccharides, and materials that have undergone some degree of processing, such as cellulose fibers.

“Biodegradable” materials are defined as materials whose physical and chemical properties undergo deterioration and completely degrade when exposed to the environment. This property, therefore, relates to the end-of-life of the material. Bio-based materials can be biodegradable or non-degradable. Similarly, while many bio-based materials are biodegradable (e.g., starch), not all biodegradable materials are bio-based.

In context of the present invention, a “biodegradable” ingredient, or a “biodegradable” material in general, for instance a shell material, is a material which meets the pass criteria for “inherently biodegradable” and/or “readily biodegradable” in at least one OECD biodegradation study. In order to avoid any ambiguity, this means that if an ingredient passes one test but fails one or more other ones, the pass result overrules the other test results.

Enzymes are proteins that act as biological catalysts by accelerating chemical reactions.

Enzyme immobilization can be defined as the confinement of enzyme molecules onto/within a support, physically or chemically or both, in such a way that it retains its full activity or most of its activity.

The molecules upon which enzymes act are called substrates.

The prefix “poly” (such as in “polycarboxylic”, “polyol” aso) denotes a functionality ≥2.

The term “amino acid ester derivative” refers to any derivative of an amino acid ester resulting from reaction at an amino group, ester group, side-chain functional group, or from the replacement of any hydrogen by a heteroatom.

The term “amino acid derivative” refers to any derivative of an amino acid resulting from reaction at an amino group, carboxy group, side-chain functional group, or from the replacement of any hydrogen by a heteroatom.

### DETAILED DESCRIPTION

Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

The applicant has surprisingly and unexpectedly found that microcapsules can be obtained by in situ enzymatic polymerization processes starting from various substrates. In particular, the applicant found that polymerization reactions can take place in an oil-in-water emulsion medium, wherein the oil droplets become encapsulated by the reaction product of the enzymatically catalyzed polymerization of the substrate. The encapsulating material obtained in this way is novel and is suitable for use as an encapsulating medium.

Enzymatic reactions usually occur under mild conditions, they reduce resource consumption and waste generation compared to the traditional production schemes. Enzyme-catalyzed polymerization (enzymatic polymerization) in particular affords novel polymeric materials which are often difficult or even impossible to be synthesized by conventional polymerization processes.

The invention, therefore, provides a microcapsule composition comprising at least one core-shell microcapsule, wherein the at least one core-shell microcapsule comprises a hydrophobic core and a shell surrounding the core, wherein the shell comprises a polymer or oligomer generated in situ by an enzyme-catalysed oligomerization/polymerization of a substrate.

It is advantageous if the substrate is derived from nature (bio-based), sustainable and/or renewable, giving rise to a bio-based encapsulating material.

Most of these substrates as well as the resulting oligomers and polymers are also biodegradable, providing an additional benefit.

### Hydrophobic Core

The hydrophobic core comprises a solvent material, at least one benefit agent, or a mixture thereof.

### Benefit Agent

Suitable benefit agents to be incorporated into the core of the core-shell microcapsules of the present invention include perfume/fragrance ingredients, flavor ingredients, cosmetic ingredients, bioactive agents (such as bactericides, insect repellents and pheromones), substrate enhancers (such as silicones and brighteners), enzymes (such as esterases, proteases and oxido-reductases), dyes, pigments and nutraceuticals.

In one embodiment, the at least one benefit agent may be at least one fragrance ingredient. A comprehensive list of fragrance ingredients that may be encapsulated in accordance with the present invention may be found in the perfumery literature, for example “Perfume & Flavor Chemicals”, S. Arctander (Allured Publishing, 1994). Encapsulated fragrance ingredients according to the present invention preferably comprise fragrance ingredients selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER CORE1-((2-(3,8,8,11a-tetramethyldodecahydro-1H-(tert-butyl)cyclohexyl)oxy) butan-2-olAMBERKETAL 3,5a-epoxynaphtho[2,1-c]oxepine); AMBERMAX (1,3,4,5,6,7-hexahydro-.beta., 1,1,5,5-pentamethyl-2H-2,4a-Methanonaphthalene-8-ethanol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYL SALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-methylenebicyclo[3.3.1]nonane); BORNEOL CRYSTALS ((1S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl) propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl) quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4 (5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl) hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-NITRILE (3,7-dimethyloct-6-enenitrile); CLONAL en-1-yl formate); CITRONELLYL (dodecanenitrile); CORANOL (4-cyclohexyl-2-methylbutan-2-ol); COSMONE ((Z)-3-methylcyclotetradec-5-enone); COUMARIN PURE CRYSTALS (2H-chromen-2-one); CRESYL ACETATE PARA ((4-methylphenyl)acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); CYMENE PARA (1-methyl-4-propan-2-ylbenzene); DAMASCENONE ((E)-1-(2,6,6-trimethylcyclohexa-1,3-dien-1-yl) but-2-en-1-one); DAMASCONE ALPHA ((E)-1-(2,6,6-trimethylcyclohex-2-en-1-yl) but-2-en-1-one); DAMASCONE DELTA (1-(2,6,6-trimethyl-1-cyclohex-3-enyl) but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE (3-methyl-2-pentylcyclopent-2-enone); DIHYDRO MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5 (6H)-ylidene) butanal); EBANOL ((E)-3-methyl-5-(2,2,3-trimethylcyclopent-3-en-1-yl) pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl) ethanone); FLORALOZONE (3-(4-ethylphenyl)-2,2-dimethylpropanal); FLORHYDRAL (3-(3-isopropylphenyl) butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl) pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HELIOTROPINE CRYSTALS (benzo[d][1,3]dioxole-5-carbaldehyde); HERBANATE ((2S)-ethyl 3-isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (1H-indole); INDOLENE (8,8-di(1H-indol-3-yl)-2,6-dimethyloctan-2-ol); IONONE BETA ((E)-4-(2,6,6-trimethylcyclohex-1-en-1-yl) but-3-en-2-one); IRISANTHEME ((E)-3-methyl-4-(2,6,6-trimethylcyclohex-2-en-1-yl) but-3-en-2-one); IRISONE ALPHA ((E)-4-(2,6,6-trimethylcyclohex-2-en-1-yl) but-3-en-2-one); IRONE ALPHA ((E)-4-(2,5,6,6-tetramethylcyclohex-2-en-1-yl) but-3-en-2-one); ISO E SUPER (1-(2,3,8,8-tetramethyl-1,2,3,4,5,6,7,8-octahydronaphthalen-2-yl) ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl) phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl) but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl) methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy) hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); #N/ALINALOOL (3,7-dimethylocta-1,6-dien-3-ol); LINALOOL OXIDE (2-(5-methyl-5-vinyltetrahydrofuran-2-yl) propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl) methanol); MEFROSOL (3-methyl-5-phenylpentan-1-ol); MELONAL (2,6-dimethylhept-5-enal); #N/A #N/AMERCAPTO-8-METHANE-3-ONE (mercapto-para-menthan-3-one); METHYL ANTHRANILATE (methyl 2-aminobenzoate); METHYL BENZOATE (methyl (1-((1S,8aS)-1,4,4,6-tetramethyl-2,3,3a,4,5,8-benzoate); METHYL CEDRYL KETONE hexahydro-1H-5,8a-methanoazulen-7-yl) ethanone); METHYL CINNAMATE (methyl 3-phenylprop-2-enoate); METHYL DIANTILIS (2-ethoxy-4-(methoxymethyl) phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl) propyl)cyclopentanone); NEOBERGAMATE FORTE (2-methyl-6-methyleneoct-7-en-2-yl acetate); NEOCASPIRENE EXTRA (10-isopropyl-2,7-dimethyl-1-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7,11-trimethyldodeca-1,6,10-trien-3-yl acetate); NEROLINE CRYSTALS (2-ethoxynaphthalene); NEROLIONE (1-(3-methylbenzofuran-2-yl) ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclo[3.1.1]hept-2-en-2-yl)ethyl acetate); NYMPHEAL (3-(4-(2-methylpropyl)-2-methylphenyl) propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl) butanamide); PEACH PURE (5-heptyldihydrofuran-2 (3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl) acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-trimethylbicyclo[3.1.1]hept-2-ene); PINENE BETA (6,6-dimethyl-2-methylenebicyclo[3.1.1]heptane); PINOACETALDEHYDE (3-(6,6-dimethylbicyclo[3.1.1]hept-2-en-2-yl) propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2 (3H)-one); RADJANOL SUPER ((E)-2-ethyl-4-(2,2,3-trimethylcyclopent-3-en-1-yl) but-2-en-1-ol); RASPBERRY KETONE (4-(4-hydroxyphenyl) butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE EXTRA (3-methyl-5-(2,2,3-trimethylcyclopent-3-en-1-yl) pentan-2-ol); SCENTAURUS CLEAN (ethyl(Z)-2-acetyl-4-methyltridec-2-enoate); SCENTAURUS JUICY (4-(dodecylthio)-4-methylpentan-2-one); SERENOLIDE (2-(1-(3,3-dimethylcyclohexyl) ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl) pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl) propan-2-ol); TERPINEOL ALPHA (2-(4-methyl-1-cyclohex-3-enyl) propan-2-ol); TERPINEOL PURE (2-(4-methylcyclohex-3-en-1-yl) propan-2-ol); TERPINOLENE (1-methyl-4-(propan-2-ylidene)cyclohex-1-ene); TERPINYL ACETATE (2-(4-methyl-1-cyclohex-3-enyl) propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

In particularly preferred embodiments of the present invention, more than 75%, preferably more than 80%, even more preferably more than 85%, even still more preferably more than 90%, even yet still more preferably more than 95%, of the fragrance ingredients are biodegradable and selected from ACETYL ISOEUGENOL ((E)-2-methoxy-4-(prop-1-en-1-(2,6,10-trimethylundec-9-enal); AGRUMEX (2-(tert-yl)phenyl acetate); ADOXAL butyl)cyclohexyl acetate); ALDEHYDE C 10 DECYLIC (decanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA (2-methylundecanal); ALDEHYDE C 8 OCTYLIC (octanal); CYCLAMEN ALDEHYDE EXTRA (3-(4-isopropylphenyl)-2-methylpropanal); ALDEHYDE ISO C 11 ((E)-undec-9-enal); ALLYL AMYL GLYCOLATE (prop-2-enyl 2-(3-methylbutoxy)acetate); ALLYL CYCLOHEXYL PROPIONATE (prop-2-enyl 3-cyclohexylpropanoate); ALLYL OENANTHATE (prop-2-enyl heptanoate); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL SALICYLATE (pentyl 2-hydroxybenzoate); AUBEPINE PARA CRESOL (4-methoxybenzaldehyde); BENZYL ACETATE (benzyl acetate); BENZYL SALICYLATE (benzyl 2-hydroxybenzoate); BORNYL ACETATE ((2S,4S)-1,7,7-trimethylbicyclo[2.2.1]heptan-2-yl acetate); CARVACROL (5-isopropyl-2-methylphenol); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CITRAL ((E)-3,7-dimethylocta-2,6-dienal); CITRONELLOL (3,7-dimethyloct-6-en-1-ol); CITRONELLYL ACETATE (3,7-dimethyloct-6-en-1-yl acetate); COSMONE ((Z)-3-methylcyclotetradec-5-enone); CRESYL METHYL ETHER PARA (1-methoxy-4-methylbenzene); CYCLOHEXYL ETHYL ACETATE (2-cyclohexylethyl acetate); CYCLOHEXYL SALICYLATE (cyclohexyl 2-hydroxybenzoate); DAMASCENONE ((E)-1-(2,6,6-trimethylcyclohexa-1,3-dien-1-yl) but-2-en-1-one); DAMASCONE ALPHA ((E)-1-(2,6,6-trimethylcyclohex-2-en-1-yl) but-2-en-1-one); DECALACTONE GAMMA (5-hexyloxolan-2-one); DECENAL-4-TRANS ((E)-dec-4-enal); DIHYDRO MYRCENOL (2,6-dimethyloct-7-en-2-ol); DIPHENYL OXIDE (oxydibenzene); DIHYDRO ANETHOLE (1-methoxy-4-propylbenzene); DIHYDRO JASMONE (3-methyl-2-pentylcyclopent-2-enone); DIMETHYL ANTHRANILATE (methyl 2-(methylamino)benzoate); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETOL (2,6-dimethylheptan-2-ol); DODECALACTONE DELTA (6-heptyltetrahydro-2H-pyran-2-one); DODECALACTONE GAMMA (5-octyloxolan-2-one); DODECENAL ((E)-dodec-2-enal); EBANOL ((E)-3-methyl-5-(2,2,3-trimethylcyclopent-3-en-1-yl) pent-4-en-2-ol); ETHYL HEXANOATE (ethyl hexanoate); ETHYL METHYL-2-BUTYRATE (ethyl 2-methyl butyrate); ETHYL MALTOL (2-ethyl-3-hydroxy-4H-pyran-4-one); ETHYL OENANTHATE (ethyl heptanoate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FIXAMBRENE (3a,6,6,9a-tetramethyldodecahydronaphtho[2,1-b]furan); FLORHYDRAL (3-(3-isopropylphenyl) butanal); FLORIDILE ((E)-undec-9-enenitrile); GALBANONE PURE (1-(5,5-dimethylcyclohex-1-en-1-yl) pent-4-en-1-one); GARDENOL (1-phenylethyl acetate); GERANIOL ((E)-3,7-dimethylocta-2,6-dien-1-ol); GERANYL ACETATE ((E)-3,7-dimethylocta-2,6-dien-1-yl acetate); HABANOLIDE ((E)-oxacyclohexadec-12-en-2-one); HEDIONE (methyl 3-oxo-2-pentylcyclopentaneacetate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS SALICYLATE ((Z)-hex-3-en-1-yl 2-hydroxybenzoate); HEXYL ACETATE (hexyl acetate); INDOLENE (8,8-di(1H-indol-3-yl)-2,6-dimethyloctan-2-ol); IONONE BETA ((E)-4-(2,6,6-trimethylcyclohex-1-en-1-yl) but-3-en-2-one); IRISANTHEME ((E)-3-methyl-4-(2,6,6-trimethylcyclohex-2-en-1-yl) but-3-en-2-one); IRISONE ALPHA ((E)-4-(2,6,6-trimethylcyclohex-2-en-1-yl) but-3-en-2-one); ISOAMYL ACETATE (3-methylbutyl acetate); ISOAMYL BUTYRATE (3-methylbutyl butanoate); ISOEUGENOL ((E)-2-methoxy-4-(prop-1-en-1-yl) phenol); ISOJASMONE B 11 (2-hexylcyclopent-2-en-1-one); ISORALDEINE ((E)-3-methyl-4-(2,6,6-trimethylcyclohex-2-en-1-yl) but-3-en-2-one); JASMONYL (3-butyl-5-methyltetrahydro-2H-pyran-4-yl acetate); LAITONE (8-isopropyl-1-oxaspiro[4.5]decan-2-one); LEMONILE ((2E,6Z)-3,7-dimethylnona-2,6-dienenitrile); LINALOOL (3,7-dimethylocta-1,6-dien-3-ol); LINALOOL OXIDE (2-(5-methyl-5-vinyltetrahydrofuran-2-yl) propan-2-ol); LINALYL ACETATE (3,7-dimethylocta-1,6-dien-3-yl acetate); MANZANATE (ethyl 2-methylpentanoate); MAYOL ((4-isopropylcyclohexyl) methanol); MEFROSOL (3-methyl-5-phenylpentan-1-ol); MELONAL (2,6-dimethylhept-5-enal); MERCAPTO-8-METHANE-3-ONE (mercapto-para-menthan-3-one); METHYL ANTHRANILATE (methyl 2-aminobenzoate); METHYL BENZOATE (methyl benzoate); METHYL DIANTILIS (2-ethoxy-4-(methoxymethyl) phenol); METHYL HEPTENONE PURE (6-methylhept-5-en-2-one); METHYL LAITONE (8-methyl-1-oxaspiro[4.5]decan-2-one); METHYL OCTYNE CARBONATE (methyl non-2-ynoate); METHYL SALICYLATE (methyl 2-hydroxybenzoate); NECTARYL (2-(2-(4-methylcyclohex-3-en-1-yl) propyl)cyclopentanone); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLINE CRYSTALS (2-ethoxynaphthalene); NEROLIONE (1-(3-methylbenzofuran-2-yl) ethanone); NERYL ACETATE ((Z)-3,7-dimethylocta-2,6-dien-1-yl acetate); NONADIENAL ((2E,6Z)-nona-2,6-dienal); NONENAL-6-CIS ((Z)-non-6-enal); NONENOL-6-CIS ((Z)-non-6-en-1-ol); NYMPHEAL (3-(4-(2-methylpropyl)-2-methylphenyl) propanal); OCTALACTONE DELTA (6-propyltetrahydro-2H-pyran-2-one); ORANGER CRYSTALS (1-(2-naphtalenyl)-ethanone); PARA TERT BUTYL CYCLOHEXYL ACETATE (4-(tert-butyl)cyclohexyl acetate); PEACH PURE (5-heptyldihydrofuran-2 (3H)-one); PELARGOL (3,7-dimethyloctan-1-ol); PHENYL ETHYL ACETATE (2-phenylethyl acetate); PINENE ALPHA (2,6,6-trimethylbicyclo[3.1.1]hept-2-ene); PINENE BETA (6,6-dimethyl-2-methylenebicyclo[3.1.1]heptane); POMAROSE ((2E,5E)-5,6,7-trimethylocta-2,5-dien-4-one); POMELOL FF (2,4,7-Trimethyl-6-octen-1-ol); PRENYL ACETATE (3-methylbut-2-en-1-yl acetate); PRUNOLIDE (5-pentyldihydrofuran-2 (3H)-one); RASPBERRY KETONE (4-(4-hydroxyphenyl) butan-2-one); ROSALVA (dec-9-en-1-ol); ROSE OXIDE CO (4-methyl-2-(2-methylprop-1-en-1-yl)tetrahydro-2H-pyran); ROSYRANE SUPER (4-methyl-2-phenyl-3,6-dihydro-2H-pyran); SAFRANAL (2,6,6-trimethylcyclohexa-1,3-dienecarbaldehyde); SCENTAURUS JUICY (4-(dodecylthio)-4-methylpentan-2-one); SILVIAL (2-methyl-3-[4-(2-methylpropyl)phenyl]propanal); STYRALLYL ACETATE (1-phenylethyl acetate); SYLKOLIDE ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINEOL (2-(4-methylcyclohex-3-en-1-yl) propan-2-ol); TERPINOLENE (1-methyl-4-(propan-2-ylidene)cyclohex-1-ene); TETRAHYDRO LINALOOL (3,7-dimethyloctan-3-ol); TOSCANOL (1-(cyclopropylmethyl)-4-methoxybenzene); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); UNDECAVERTOL ((E)-4-methyldec-3-en-5-ol); YARA YARA (2-methoxynaphtalene); BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These ingredients have the advantage of providing microcapsules which are particularly sustainable.

The at least one benefit agent may comprise at least one fragrance precursor, meaning a material that is capable of releasing a fragrance ingredient by the means of a stimulus, such as a change of temperature, the presence of oxidants, the action of enzymes or the action of light. Such fragrance precursors are well-known to the art.

The at least one benefit agent may also comprise at least one functional cosmetic ingredient. The functional cosmetic ingredients for use in the encapsulated composition are preferably hydrophobic. Preferably, the cosmetic ingredients have a calculated octanol/water partition coefficient (ClogP) of 1.5 or more, more preferably 3 or more. Alternatively preferred, the ClogP of the cosmetic ingredient is from 2 to 7.

Particularly useful functional cosmetic ingredients may be selected from the group consisting of emollients, smoothening ingredients, hydrating ingredients, soothing and relaxing ingredients, decorative ingredients, deodorants, anti-aging ingredients, cell rejuvenating ingredients, draining ingredients, remodeling ingredients, skin levelling ingredients, preservatives, anti-oxidants, antibacterial or bacteriostatic ingredients, cleansing ingredients, lubricating ingredients, structuring ingredients, hair conditioning ingredients, whitening ingredients, texturing ingredients, softening ingredients, anti-dandruff ingredients, and exfoliating ingredients.

Particularly useful functional cosmetic ingredients include, but are not limited to hydrophobic polymers, such as alkyldimethylsiloxanes, polymethylsil-sesquioxanes, polyethylene, polyisobutylene, styrene-ethylene-styrene and styrene-butylene-styrene block copolymers, and the like; mineral oils, such as hydrogenated isoparaffins, silicone oils and the like; vegetable oils, such as argan oil, jojoba oil, aloe vera oil, and the like; fatty acids and fatty alcohols and their esters; glycolipides; phospholipides; sphingolipides, such as ceramides; sterols and steroids; terpenes, sesquiterpenes, triterpenes and their derivatives; essential oils, such as Arnica oil, Artemisia oil, Bark tree oil, Birch leaf oil, Calendula oil, Cinnamon oil, Echinacea oil, Eucalyptus oil, Ginseng oil, Jujube oil, Helianthus oil, Jasmine oil, Lavender oil, Lotus seed oil, Perilla oil, Rosmary oil, Sandal wood oil, Tea tree oil, Thyme oil, Valerian oil, Wormwood oil, Ylang Ylang oil, and Yucca oil.

In particular, the at least one functional cosmetic ingredient may be selected from the group consisting of Sandal wood oil, such as Fusanus Spicatus kernel oil; Panthenyl triacetate; Tocopheryl acetate; Tocopherol; Naringinin; Ethyl linoleate; Farnesyl acetate; Farnesol; Citronellyl methyl crotonate; and Ceramide-2 (1-Stearoiyl-C18-Sphingosine, CAS-No: 100403-19-8).

The at least one benefit agent may comprise agents which suppress or reduce malodour and its perception by adsorbing odour, agents which provide a warming or cooling effect, insect repellents or UV absorbers.

### Solvent Materials

The present invention also contemplates the incorporation of solvent materials instead or in addition to the benefit agent. The solvent materials are hydrophobic materials that are miscible in the benefit agents used in the present invention. Suitable solvent materials are those having reasonable affinity for the benefit agents and a Clog P greater than 2.5, preferably greater than 6 and most preferably greater than 10. Suitable solvent materials include, but are not limited to triglyceride oil, mono and diglycerides, mineral oil, silicone oil, diethyl phthalate, polyalpha olefins, castor oil and isopropyl myristate.

Examples of suitable solvent materials are mono-, di- and tri-esters, and mixtures thereof, of fatty acids and glycerine, optionally wherein the fatty acid chain ranges from C4-C26, optionally wherein the fatty acid chain have any level of unsaturation; isopropyl myristate; fatty acid esters of polyglycerol oligomers: R²CO—[OCH₂—CH(OCOR¹)—CH₂O—]ₙ, where R¹ and R² can be H or C4-C26 aliphatic chains, or mixtures thereof, and n ranges between 2-50, preferably 2-30; nonionic fatty alcohol alkoxylates, optionally wherein the alkoxy group atre ethoxy, propoxy, butoxy, or mixtures thereof; di- and tri-fatty acid chain containing nonionic, anionic and cationic surfactants, and mixtures thereof; fatty acid esters of polyethylene glycol, polypropylene glycol, and polybutylene glycol, or mixtures thereof; polyalphaolefins; mineral oil; silicone oils such as polydimethyl siloxane and polydimethylcyclosiloxane; diethyl phthalate or di-isodecyl adipate.

In one embodiment the core comprises solvent materials combined with at least one benefit agent. In one embodiment, the benefit agent is a fragrance material.

In one embodiment, the benefit agent is bio-degradable.

### Polymer or Oligomer

The polymer or oligomer comprised in the shell of the core-shell microcapsules of the present invention is generated in situ by an enzyme-catalysed process.

Oligomers and polymers that are produced from renewable carbon sources and contain components extracted from plant biomass, for example, starch, cellulose, vegetable oil, lignin from plant or wood pulp, obtained by photosynthesis from atmospheric carbon dioxide, as well as products of microbial and animal nature are all bio-based. Most of these materials are also expected to be biodegradable.

In one embodiment, the macromolecular materials (oligomers or polymers) generated by the in-situ enzymatic polymerization comprise oligo- or polyesters, oligo- or polyamides, oligo-or poly(amino acid) s, oligo- or polyaromatics or mixtures thereof.

### Enzymes

Enzymes have been applied in many industrial (organic) processes. According to the Enzyme Commission (EC), all the enzymes are classified into six main groups:
EC1: Oxido-reductases, which catalyze redox-reactions by electron transfer.
EC2: Transferases, which catalyze the transfer of a functional group, for example a methyl group or a glycosyl group, from one compound (donor) to another compound (acceptor).
EC3: Hydrolases, which catalyze the hydrolysis of various bonds in order to transfer functional groups to water.
EC4: Lyases, which catalyze the cleavage of C—C, C—O, C—N and other bonds by means other than hydrolysis or oxidation.
EC5: Isomerases, which catalyze either racemization or epimerization of chiral centres; isomerases are subdivided according to their substrates.
EC6: Ligases, which catalyze the coupling of two molecules with concomitant hydrolysis of the diphosphate-bond in ATP or a similar triphosphate.

Mechanisms of enzyme catalysis vary, but are all similar in principle to other types of chemical catalysis in that the crucial factor is a reduction of energy barrier(s) separating the reactants (or substrates) from the products. The reduction of activation energy (Eₐ) increases the fraction of reactant molecules that can overcome this barrier and form the product. An important principle is that since they only reduce energy barriers between products and reactants, enzymes always catalyze reactions in both directions, and cannot drive a reaction forward or affect the equilibrium position-only the speed with which is it achieved. As with other catalysts, the enzyme is not consumed or changed by the reaction (as a substrate is) but is recycled such that a single enzyme performs many rounds of catalysis.

Enzymatic polymerization has been increasingly applied in the past years, providing many successful examples of functional polymeric materials. The application of enzymes in polymer chemistry has many advantages: polymerizations can be performed under mild conditions with regard to pressure, temperature and pH, which makes enzymatic reactions very energy efficient. Enzymes can be highly selective: chemo-, regio-, and enantioselectivity can all be enzymatically induced, opening a novel direction towards precision polymer synthesis. Enzymes are considered to be ‘green’, non-toxic catalysts, which can meet the increasing demands regarding commercial, ecological and biomedical requirements.

### Oxidoreductases

Most oxidoreductases contain low-valent metals as a catalytic centre. It is believed that the polymerization process involves an enzyme-initiated radical polymerization of an aromatic or vinyl monomer, therefore the enzyme does not catalyze the polymerization reaction as such, but a reaction where active forms of the monomer are formed.

For example, laccases (EC1.10.3.2) catalyze the oxidation of organic substrates, mainly derivatives of phenol and aniline, with molecular oxygen. The catalytic center of the laccase includes four copper atoms that form complexes with the imidazole groups of histidines and constitute an electron transfer chain. It is believed that one copper atom is directly involved in the oxidation of the organic reducing agent, while the other three form a center where oxygen is reduced to water.

Peroxidases (EC1.11.1) catalyze the oxidation of a variety of organic substrates with hydrogen peroxide. The catalytic site of peroxidase includes a heme with an iron atom, which has a variable oxidation state. In the catalytic cycle, it is believed that Fe (IV) species are formed at the active site and oxidize the organic substrate to polymerizing active radicals. For example, a horseradish peroxidase (HRP) in the presence of hydrogen peroxide efficiently catalyzed phenol polymerization to obtain a phenolic polymer of moderate molecular weight consisting of a mixture of phenylene and oxyphenylene units.

Oxidoreductases have been used for the polymerization of phenyl-containing compounds to obtain poly(phenols) or poly(anilines).

### Hydrolases

Hydrolases have been the most-investigated enzymes for oligo/polymer synthesis, the most widely employed in these studies being the esterases (EC3.1) and the proteases (EC3.4).

For example, carboxylic ester hydrolases (EC 3.1.1.) of the esterase category act on ester bonds and have been applied in several biotechnological processes. Examples of carboxylic ester hydrolases acting on ester bonds are triacylglycerol lipases (EC 3.1.1.3), carboxylesterases (EC 3.1.1.1.) and cutinases (EC 3.1.1.74).

Lipases (EC3.1.1.3) can catalyze a very wide range of reactions, covering not only the formation and hydrolysis of an ester bond, but also amidation, aminolysis, aldol condensation, Michael addition, opening of lactone rings, epoxidation. Lipases have been successfully employed to obtain biodegradable polymers of many types, most notably polyesters, polyamides and polyesteramides. There are two main types of polymerization reactions with lipases: ring-opening polymerization and polycondensation. Ring-opening polymerization uses ring-closed esters, such as lactones or lactides to open them and to perform propagation reactions of the chain. Polycondensation uses either hydroxy acids as monomers or diacids (diesters) and diols to link them via ester bonds.

Cutinases (EC 3.1.1.74) are capable of catalyzing ester hydrolysis, esterification and transesterification reactions. In this regard, cutinases are considered as an alternative to lipases in the context of obtaining biodegradable polymers, in particular polyesters.

Proteolytic enzymes such as proteases (EC3.4)) can act as enzymatic catalysts in the synthesis of oligopeptides and oligopeptidomimetics with various functional and physical properties. Among proteases, papain has been employed the most in enzymatic polymerization, probably because of its wide substrate specificity, stability and ease of production. Proteases have been shown to catalyze polymerization reactions of esters of amino acids and copolymerization of amino acids with polyamides.

Hydrolases have been applied to synthesize polyesters, polycarbonates, polyamides, polyamino acids, oligopeptides and polyesteramides.

Therefore, in one embodiment, the enzyme is:
a) a hydrolase (EC3), such as an esterase (EC3.1) or a protease/peptidase (EC3.4); or
b) an oxidoreductase (EC1), such as oxidoreductases that act on diphenols as donors (EC1.10) or on a peroxide as acceptor (EC1.11).

In one embodiment,
a1) the esterase is a carboxylic-ester hydrolase (EC3.1.1) such as a carboxylesterase (EC3.1.1.1), a triacylglycerol lipase (EC3.1.1.3) or a cutinase (EC3.1.1.74), preferably a triacylglycerol lipase (EC3.1.1.3) such as Amano Lipase PS;
a2) the protease/peptidase is a serine endopeptidase (EC3.4.21) or a cysteine endopeptidase (EC3.4.22) such as papain;
b) the oxidoreductase is a peroxidase (EC1.11.1), preferably a peroxidase EC1.11.1.7, such as a peroxidase from horseradish (horseradish peroxidase, HRP).

In one embodiment, the enzyme is immobilized on a solid support by covalent binding and/or physical adsorption, creating a heterogeneous immobilized enzyme system. The heterogeneity of the immobilized enzyme systems allows an easy recovery of both enzymes and products, multiple re-use of enzymes, continuous operation of enzymatic processes, rapid termination of reactions, and greater variety of bioreactor designs.

### Substrate and Oligomer/Polymer

To carry out enzymatic polymerization reactions, appropriate monomers are required.

As discussed above, a series of oligo- or polymers have been obtained by enzymatic polymerization, including polyesters, polyamides, amino acid-based polymers including oligo and polypeptides, polyphenylene/oxyphenylene-containing oligo- or polymers.

In one embodiment, the monomer is derived from nature (bio-based), is sustainable and/or renewable, giving rise to a bio-based oligomer/polymer.

Most of these substrates as well as the resulting oligomers and polymers are also biodegradable, providing an additional benefit.

Polyesters are a class of polymers bearing the ester functional group as the linkage between the individual monomers. The approach for the synthesis of polyesters entails two different pathways: (i) ring-opening polymerization of cyclic monomers (lactones, cyclic diesters, cyclic carbonates and cyclic ketene acetals), and (ii) step-growth polycondensation of either diacids or diesters with diols or polyalcohols (polyols), or self-polycondensation of hydroxyacids or hydroxyesters.

Generally speaking, lactones, diacids and their ester and anhydride derivatives, diols, polyols, cyclic carbonates and hydroxyacids and their esters are suitable building blocks for polyester synthesis.

Polyamides are polymers in which the monomeric units are linked together by amide bonds. Lactams, ω-amino acids and their esters, diacids and their derivatives, and diamines are suitable monomers for polyamide synthesis.

Polyaminoacids are amino acid-based polymers and are composed of amino acids as monomeric units. Peptides are formed when the amino group of one amino acid forms an amide (peptide) linkage with the carboxyl group of another amino acid. Oligopeptides consist of 2 to about 20 aminoacids and can include di-, tri-, tetra-aso peptides. Suitable substrates for obtaining polyaminoacids and/or oligopeptides are aminoacid esters and their derivatives. The amino acids used in chemoenzymatic polymerization can be classified into hydrophobic and hydrophilic amino acids. Proteases have relatively high affinity for hydrophobic amino acid derivatives. The hydrophobic nature of these amino acids makes the resulting polypeptides insoluble in water, leading to precipitation during the polymerization process. In contrast, hydrophilic amino acid derivatives with or without a protecting group on a reactive side group are also available for chemoenzymatic polymerization. The reactive groups on the side chains, such as alcohol and amine groups, can be used for various types of functionalization of the resulting polypeptides. However, because of their water solubility, an additional process for the isolation of the products from the proteases used in the synthesis reaction is required.

There is a vast amount of structurally diverse phenols, anilines and other aromatic monomers, which have been subjected to enzyme-initiated radical polymerization yielding poly(phenols) and poly(anilines).

Therefore, monomers suitable to undergo enzymatic polymerization according to the invention are selected from the group consisting of:
lactones, cyclic carbonates, diacids and their ester and anhydride derivatives, diols, polyols and hydroxyacids and their esters;
lactams, ω-amino acids and their esters, diacids and their derivatives, and diamines;
hydrophobic and hydrophilic amino acid esters and their derivatives;
phenyl-containing amino acids and peptides.

In one embodiment, the substrate is selected from the group consisting of lactones, cyclic carbonates, polycarboxylic compounds, polyols, cyclic amides/lactams, ω-amino acids and their esters, amino acid alkyl esters and their derivatives, oligopeptide alkyl esters, phenyl containing peptides, phenyl containing amino acids or combinations thereof.

In one embodiment,
a1) when the enzyme is a lipase, the substrate is selected from the group consisting of lactones, cyclic carbonates, polycarboxylic compounds, polyols, cyclic amides/lactams, w-amino acids and their esters, amino acid alkyl esters and their derivatives and combinations thereof;
a2) when the enzyme is a protease, the substrate is selected from the group consisting of amino acid alkyl esters and their derivatives, oligopeptide alkyl esters and combinations thereof;
b) when the enzyme is an oxidoreductase, the substrate is a phenyl-containing amino acid or amino-acid derivative, a phenyl-containing peptide or combinations thereof.

In one embodiment,
the lactones are macrocyclic lactones having more than 12-membered rings, such as pentadecanolide, ambrettolide, habanolide or combinations thereof;
the cyclic carbonates are cyclic carbonates having more than 8-memberered rings;
the polycarboxylic compounds are polycarboxylic compounds having more than 8 carbon atoms;
the polyols are polyols having more than 8 carbon atoms;
the cyclic amides/lactams are lactams having more than 8-membered rings;
the amino acid alkyl esters and their derivatives are hydrophobic amino acid alkyl esters, such as the alkyl esters of alanine, valine, isoleucine, leucine, methionine, phenylalanine, tyrosine, tryptophan or combinations thereof;
the oligopeptide alkyl esters are oligopeptide alkyl esters wherein the peptide comprises 2 to 9 amino acids;
the phenyl-containing amino acid is tyrosine, phenylalanine or combinations thereof;
the phenyl-containing peptide is phenyl-containing peptide wherein the peptide comprises 2 to 9 amino acids.

In one embodiment, the alkyl is selected from the group consisting of methyl, ethyl, n-propyl, i-propyl, n-butyl, i-butyl, t-butyl and benzyl.

In one embodiment, the enzyme is Amano Lipase PS and the substrate is a macrocyclic lactones having more than 12-membered rings, preferably @-pentadecanolide.

In one embodiment, the enzyme is papain and the substrate is tyrosine methyl ester or Tyrosine methyl ester and N-benzoyl-N-Tyrosine ethyl ester.

In one embodiment, the enzyme is horseradish peroxidase and the substrate is tyrosine methyl ester or Tyrosine methyl ester and N-benzoyl-N-Tyrosine ethyl ester.

In one embodiment, the microcapsule composition is in the form of an aqueous slurry.

In one embodiment, the volume average size (Dv(50)) of the microcapsules can be from 1 to 500 μm, optionally from 1 to 100 μm, preferably from 5 to 80 μm, even more preferably from 10 to 70 μm.

In one embodiment, the slurry may be dried to present the encapsulated composition in a dry powder form.

Enzymatic polymerization reactions of the present invention result in formation of oligo- or polymeric products which are capable of forming a shell surrounding benefit-agent containing oil droplets. However, some of the oligo- or polymeric products that result from the enzymatic process do not assemble to form of a shell around an oil droplet. Therefore, in one embodiment, the microcapsule composition comprises, in addition to the core-shell microcapsules encapsulating a benefit agent, oligo- or polymer residues that do not assemble to form of a shell around an oil droplet. Upon drying such a microcapsule composition, the composition comprises microcapsules encapsulating a benefit agent as well as a matrix in which a benefit agent is entrapped.

### Methods

In one aspect, a method for preparing the microcapsule composition as described herein is provided, the method comprising the steps of:
a) providing a substrate and an enzyme in an aqueous phase in conditions suitable for providing enzymatic activity;
b) providing a hydrophobic phase;
c) emulsifying the hydrophobic phase in the aqueous phase to form a microcapsule composition.

The substrate, enzyme and hydrophobic material are as described hereinabove.

In one embodiment, the method for preparing a microcapsule composition comprising at least one core-shell microcapsule, wherein the at least one core-shell microcapsule comprises a hydrophobic core and a shell surrounding the core, comprises the steps of
a) providing a substrate and an enzyme in an aqueous phase in conditions suitable for providing enzymatic activity to generate a polymer or oligomer;
b) providing a hydrophobic phase;
c) emulsifying the hydrophobic phase in the aqueous phase to form a microcapsule composition.

In one embodiment, the substrate is selected from the group consisting of lactones, cyclic carbonates, polycarboxylic compounds, polyols, cyclic amides/lactams, ω-amino acids and their esters, amino acid alkyl esters and their derivatives, oligopeptide alkyl esters, phenyl containing peptides, phenyl containing amino acids or combinations thereof.

In one embodiment, the enzyme is
a) a hydrolase (EC3), such as an esterase (EC3.1) or a protease/peptidase (EC3.4); or
b) an oxidoreductase (EC1), such as oxidoreductases that act on diphenols as donors (EC1.10) or on a peroxide as acceptor (EC1.11).

In one embodiment,
a1) the esterase is a carboxylic-ester hydrolase (EC3.1.1) such as a carboxylesterase (EC3.1.1.1), a triacylglycerol lipase (EC3.1.1.3) or a cutinase (EC3.1.1.74), preferably a triacylglycerol lipase (EC3.1.1.3) such as Amano Lipase PS;
a2) the protease/peptidase is a serine endopeptidase (EC3.4.21) or a cysteine endopeptidase (EC3.4.22) such as papain;
b) the oxidoreductase is a peroxidase (EC1.11.1), preferably a peroxidase EC1.11.1.7, such as a peroxidase from horseradish (HRP).

In one embodiment,
a1) when the enzyme is a lipase, the substrate is selected from the group consisting of lactones, cyclic carbonates, polycarboxylic compounds, polyols, cyclic amides/lactams, ω-amino acids and their esters, amino acid alkyl esters and their derivatives or combinations thereof;
a2) when the enzyme is a protease, the substrate is selected from the group consisting of amino acid alkyl esters and their derivatives, oligopeptide alkyl esters;
b) when the enzyme is an oxidoreductase, the substrate is a phenyl-containing amino acid or amino-acid derivative or a phenyl-containing peptide or combinations thereof.

In one embodiment, the hydrophobic phase comprises at least one benefit agent as defined hereinabove. In one embodiment, the at least one benefit agent is at least one fragrance ingredient. In one embodiment, the enzyme is dissolved in water, optionally in the presence of a buffer, and it is incubated at a temperature of between about 20° C. to about 50° C., preferably about 40° C. for a period of between 5 to 20 min, preferably about 10 min to activate the enzyme.

The substrate is dissolved in water, optionally in the presence of a buffer, and it is incubated at a temperature of between about 20° C. to about 50° C., preferably about 40° C. for a period of between 30 s to 2 min, preferably about 1 min.

The activated enzyme solution is added to the substrate solution to initiate the reaction.

An oil (hydrophobic) phase is added to the aqueous phase and emulsified at a temperature of between about 20° C. to about 50° C., preferably about 40° C. for a period of between 12 h to 36 h preferably about 20 h, to obtain a microcapsule composition in the form of a slurry.

In one embodiment, when the enzyme is immobilized on a solid substrate, the method includes a step of separating the reaction mixture from the immobilized enzyme on the solid substrate.

The method according to the present invention may comprise the additional step of drying the microcapsule slurry, in order to obtain a microcapsule powder.

Drying of a slurry of microcapsules is conventional, and may be carried out according techniques known in the art, such as spray-drying, evaporation, lyophilization or use of a desiccant. Typically, as is conventional in the art, dried microcapsules will be dispersed or suspended in a suitable powder, such as powdered silica, which can act as a bulking agent or flow aid. Such suitable powder may be added to the encapsulated composition before, during or after the drying step.

In particular, the drying process may be accompanied by an additional entrapping process, wherein additional benefit agent is entrapped in an entrapping material. For example, the slurry to be dried may comprise, in addition to the core-shell microcapsules obtained in the process according to the present invention, at least one non-encapsulated benefit agent and at least one entrapping material, so that the benefit agent that is not encapsulated in the core-shell microcapsule is entrapped in the entrapping material during drying. Therefore, upon drying, a matrix is formed around or coexisting with the core-shell microcapsules.

### Consumer Product

The present invention also relates to a consumer product comprising a microcapsule composition as described hereinabove. The consumer product may be selected from the group consisting of household (home) care, personal care, fabric care and pet care products.

Suitable home care products include hard surface cleaners, heavy duty detergents and detergent powders, air care compositions.

Suitable personal care products include cleansing compositions (such as shampoos, bath and shower gels, liquid soaps, soap bars), conditioning compositions (such as hair care conditioners), bath and shower lotions, oral care compositions, deodorant compositions, antiperspirant compositions, skin care products

Suitable fabric care compositions include laundry care detergents, laundry care conditioners, fabric refreshers, scent boosters.

The microcapsule composition of the present invention, presented in the form of a slurry of microcapsules suspended in an aqueous suspending medium may be incorporated as such in a consumer product base or it may be incorporated in dry powder form.

Yet another aspect of the present invention relates to the use of a microcapsule composition as described hereinabove to improve the perception or enhance the performance of the benefit agent in a consumer product.

The present invention is further illustrated by means of the following non-limiting examples.

The solid content of the microparticles has been measured by a Halogen Moisture Analyzer Mettler Toledo instrument. The test measures the encapsulation efficacy, i.e. the percentage of the theoretical fragrance that was encapsulated.

Dv(50) represents the maximum particle diameter below which 50% of the sample volume exists, also known as the median particle size by volume. It is also known as the Malvern volume weighted particle size distribution, measured using light scattering techniques.

The microscopic images were recorded with an Olympus BX51 Microscope.

The SEM images were recorded with a Jeol JSM_6010PLUS/LV Scanning electron microscope.

Table 1 shows the composition of Fragrance 1 used in the examples:

| | Fragrance 1 |
|---|---|
| | % |
| MIGLYOL 840 | 8 |
| BUTYL CYCLOHEXYL | 17 |
| ACETATE PARA | |
| FLOROCYCLENE | 4. |
| AGRUMEX | 15. |
| DAMASCONE DELTA | 4. |
| EUCALYPTUS GLOBULUS | 5. |
| OIL CHINA COSMOS | |
| ROSE OXIDE CO | 0.20 |
| ETHYL METHYL-2- | 0.50 |
| BUTYRATE | |
| PATCHOULI OIL DECOL | 2.50 |
| INDONESIA ORPUR | |
| PETALIA | 5 |
| AMYL SALICYLATE | 9. |
| TETRAHYDRO LINALOOL | 29.80 |

Table 2 shows the composition of Fragrances 2-4 used in the examples:

| | Fragrance | Fragrance | Fragrance |
|---|---|---|---|
| | 2 | 3 | 4 |
| | % | % | % |
| ACET HEXYLE | 3.00 | 4.00 | 3.43 |
| ACET P T BUTYL CYCLOHEXYLE | 12.50 | 16.67 | 0.00 |
| ACET PHENYL ETHYLE | 0.50 | 0.67 | 0.57 |
| AGRUMEX | 15.17 | 20.22 | 17.33 |
| ALD C 10 DECYLIQUE | 0.50 | 0.67 | 0.57 |
| ALD C 12 MNA PUR | 2.50 | 3.33 | 2.86 |
| ALLYL AMYL GLYCOLATE | 0.50 | 0.67 | 0.57 |
| ANTHRANILATE METHYLE EXTRA | 0.25 | 0.33 | 0.29 |
| BOISAMBRENE FORTE | 1.00 | 1.33 | 1.14 |
| DAMASCONE DELTA | 1.00 | 1.33 | 1.14 |
| DIHYDRO EUGENOL MEF | 0.20 | 0.27 | 0.23 |
| DIHYDRO JASMONE | 0.20 | 0.27 | 0.23 |
| DIHYDRO MYRCENOL | 8.00 | 10.67 | 9.14 |
| DIPHENYL OXIDE | 2.00 | 2.67 | 2.29 |
| EBANOL | 0.50 | 0.67 | 0.57 |
| ETHYL VANILLINE | 0.50 | 0.67 | 0.57 |
| EUCALYPTOL NATUREL | 3.00 | 4.00 | 3.43 |
| INDOLE PUR | 0.02 | 0.03 | 0.02 |
| IONONE BETA | 1.00 | 1.33 | 1.14 |
| ISO E SUPER | 2.50 | 3.33 | 2.86 |
| ISOPROPYL QUINOLEINE | 0.01 | 0.01 | 0.01 |
| JASMACYCLENE | 25.00 | 0.00 | 28.57 |
| KOAVONE | 3.00 | 4.00 | 3.43 |
| METHYL-2-BUTYRATE ETHYLE | 1.00 | 1.33 | 1.14 |
| NYMPHEAL | 1.00 | 1.33 | 1.14 |
| OCTINE CARBONATE METHYLE | 0.05 | 0.07 | 0.06 |
| P CRESOL PUR 10%/TEC | 0.01 | 0.01 | 0.01 |
| PATCHOULI ESS DECOL INDONESIE | 0.50 | 0.67 | 0.57 |
| ORPUR | | | |
| ROSYRANE SUPER | 0.05 | 0.07 | 0.06 |
| SALICYLATE METHYLE | 0.05 | 0.07 | 0.06 |
| TETRAHYDRO LINALOL | 12.50 | 16.67 | 14.29 |
| UNDECAVERTOL | 2.00 | 2.67 | 2.29 |

### Example 1: Synthesis of Poly(Alanine) (Poly(Ala)) Using Papain

In a 15 ml glass bottle, lyophilized papain (10 mg) was dissolved and activated in 1 mL of 1 M phosphate buffer (pH 7.0) with 5 mM Cysteine and 1 mM EDTA at a concentration of 10 mg/mL, then it was incubated at 40° C. for 10 min.

The polymerization reaction was carried out in a final volume of 7 mL. 0.75 g (0.7 M) L-Alanine ethyl ester (L-Ala-Et), 1 mL of papain (10 mg/mL), and 6 mL of 1M phosphate buffer were added to a glass reaction tube. The reaction was gently stirred at 250 rpm at 40° C. for 24 h. The product was purified by centrifugation at 7830 rpm, 25° C., for 15 min. The culot PolyAla was washed once with dilute HCl (2% v/v) and twice with distilled water. The culot was lyophilized for yield calculation (yield=71.0%).

The yield was calculated based on the amount of the starting material (monomer), the amount of released by-product (ethanol in this case) and the amount of product precipitated:

$$Y = \frac{{m}^{precipitated product}}{{m}^{monomer} - {m}^{by product}} = \frac{{m}^{precipitated product}}{{M}^{monomer} * {n}^{monomer} - {M}^{by product} * {n}^{monomer}}$$

m=mass; M=molecular mass; n=number of mol

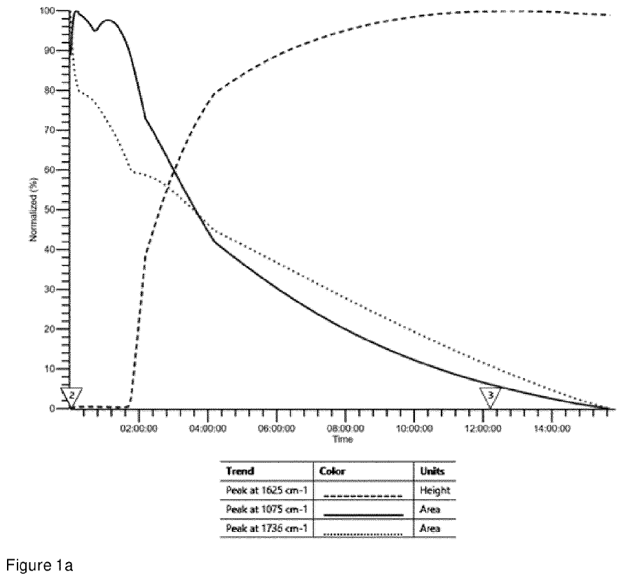

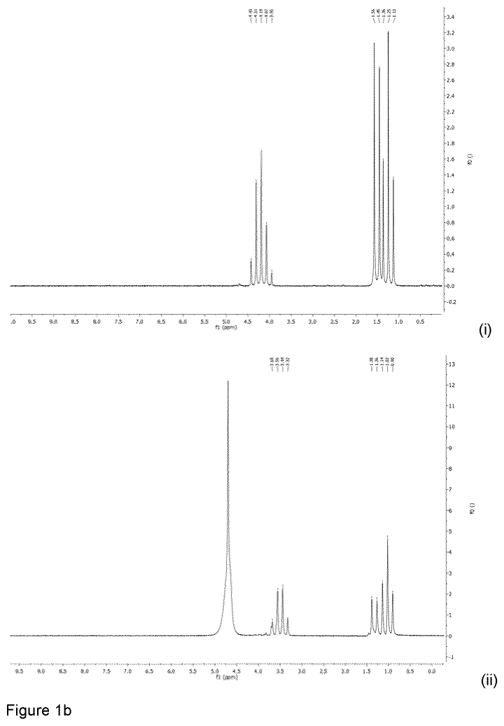

FIG. 1a shows the evolution of the peak intensity of the relevant IR wavelengths of the amine, ester and amide functional groups in the reaction medium over the course of this reaction (i.e. the reaction kinetics), demonstrating the total consumption of the starting material L-Alanine ethyl ester (containing amine and ester functionalities) and formation of amide bonds in the reaction product, thereby indicating the formation of oligo- or polypeptides.

FIG. 1b shows a comparison between the ¹H NMR (H₂O) spectra of (1bi) L-alanine ethyl ester and (1bii) Poly(Ala) confirming the consumption of the substrate and formation of polyalanine.

### Example 2: Synthesis of Poly(Tyrosine) (Poly(Tyr)) Using Papain

In a 15 ml glass bottle, lyophilized papain was dissolved and activated in 1 mL of 1 M phosphate buffer (pH 7.0) with 5 mM Cysteine and 1 mM EDTA at a concentration of 10 mg/mL, then it was incubated at 40° C. for 10 min.

The polymerization reaction was carried out in a final volume of 7 mL. 0.13 g (0.1 M) L-tyrosine methyl ester, 1 mL of papain (10 mg/mL), and 6 mL of 1M phosphate buffer were added to a glass reaction tube. The reaction was gently stirred at 250 rpm at 40° C. for 24 h. The product was purified by centrifugation at 7830 rpm, 25° C., for 15 min. The culot was washed once with dilute HCl (2% v/v) and twice with distilled water. The culot was then lyophilized for yield calculation (yield=86.8%).

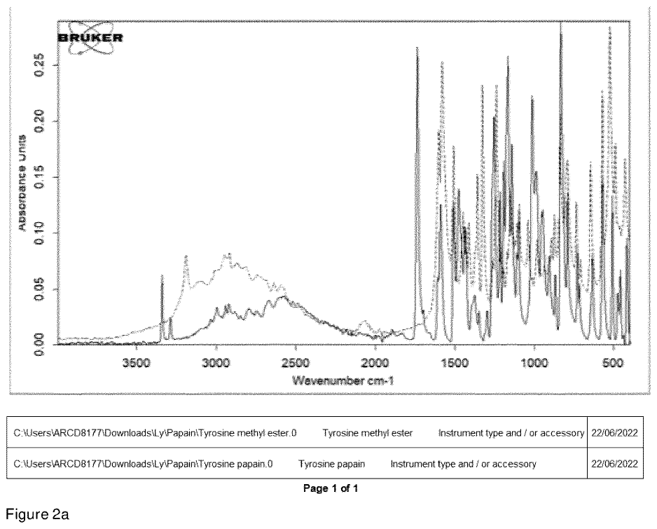

FIG. 2a shows the overlayed IR spectra of tyrosine methyl ester and Poly(Tyr), confirming the conversion of the starting monomer into a reaction product. In particular, the disappearance of the peak at 1730 cm-1, characteristic of the ester bond in the monomer, can be observed. The ester peaks are being replaced by peaks characteristic to an amide functionality in the product.

FIG. 2b shows a comparison between the ¹H NMR (H₂O) spectra of (2bi) L-tyrosine methyl ester and (2bii) the reaction product, confirming the consumption of the substrate and formation of polytyrosine.

### Example 3: Synthesis of Microcapsules Using Alanine Ethyl Ester and Papain—Mygliol

In a flask, 30 mg papain was dissolved in 2 ml phosphate buffer containing 1 mM EDTA+5 mM Cysteine. The solution was incubated at 40° C. for 10 min.

In another flask equipped with mechanical stirrer and a reflux condenser, 0.96 g Alanine ethyl ester was dispersed in 12 ml 1M phosphate buffer. The substrate solution was pre-incubated at 40° C. for 1 min, followed by addition of the activated enzyme solution to initiate the reaction. The medium was homogenized at 250 rpm for 4 h at 40° C. Then, agitation was increased up to 800 rpm. 3.5 g Mygliol (a medium chain triglyceride oil) was added into the reaction mixture. The aqueous and oil phase were combined and homogenized at 800 rpm at 40° C. The reaction was stopped after 20 h.

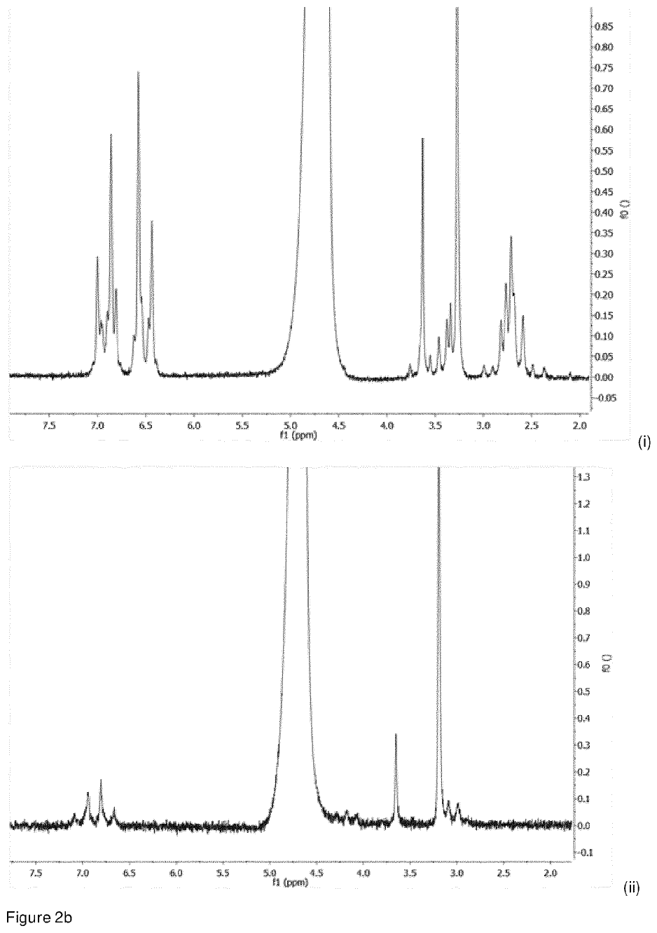

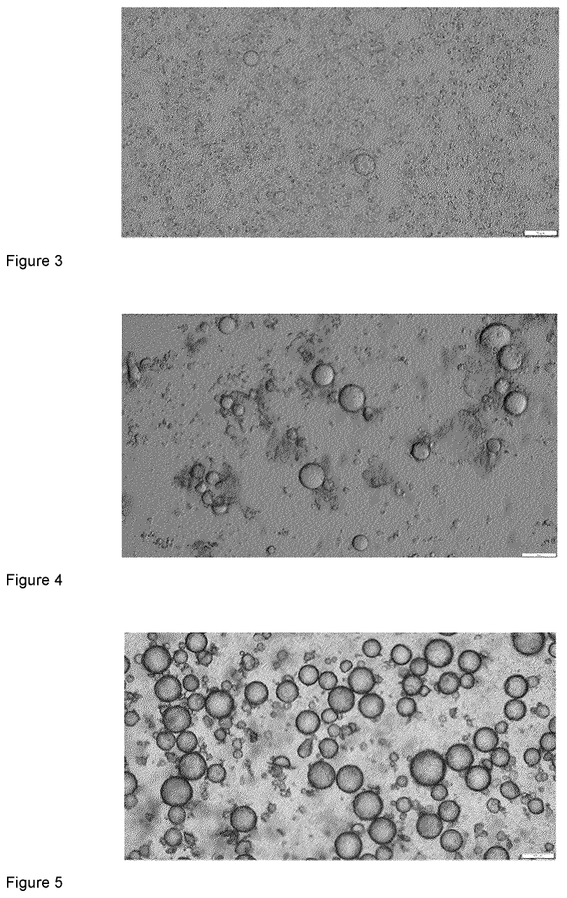

FIG. 3 illustrates a microscopic image of a microcapsule composition encapsulating Mygliol, obtained by papain-catalysed polymerization of alanine ethyl ester.

### Example 4: Synthesis of Microcapsules Using Tyrosine Methyl Ester, N-Benzoyl-N-Tyrosine Ethyl Ester (BTEE) and Papain—Fragrance 1

In a flask, 20 mg papain was dissolved in 2 ml phosphate buffer containing 1 mM EDTA+5 mM Cysteine. The solution was incubated at 40° C. for 10 min.

In another flask equipped with mechanical stirrer and a reflux condenser, 0.26 g Tyrosine methyl ester and 0.1 g BTEE was dispersed in 12 ml 1M phosphate buffer. The substrate solution was pre-incubated at 40° C. for 1 min, followed by addition of the activated enzyme solution to initiate the reaction. The medium was homogenized at 250 rpm for 4 h at 40° C. Then, agitation was increased up to 800 rpm. 3.5 g of fragrance 1 as an oil phase was added into reaction. The aqueous and oil phase were combined and homogenized at 800 rpm at 40° C. The reaction was stopped after 20 h.

FIG. 4 illustrates a microscopic image of a microcapsule composition encapsulating fragrance 1, obtained by papain-catalysed polymerization of tyrosine methyl ester and BTEE.

### Example 5: Synthesis of Microcapsules Using Tyrosine Methyl Ester, BTEE and Papain—Fragrance 2

In a flask, 20 mg papain was dissolved in 2 ml phosphate buffer containing 1 mM EDTA+5 mM Cysteine. The solution was incubated at 40° C. for 10 min.

In another flask equipped with mechanical stirrer and a reflux condenser, 0.26 g Tyrosine methyl ester and 0.1 g BTEE was dispersed in 12 ml 1M phosphate buffer. The substrate solution was pre-incubated at 40° C. for 1 min, followed by addition of the activated enzyme solution to initiate the reaction. The medium was homogenized at 250 rpm for 4 h at 40° C. Then, agitation was increased up to 800 rpm. 3.5 g of fragrance 2 as an oil phase was added into reaction. The aqueous and oil phase were combined and homogenized at 800 rpm at 40° C. The reaction was stopped after 20 h.

FIG. 5 illustrates a microscopic image of a microcapsule composition encapsulating fragrance 2, obtained by papain-catalysed polymerization of tyrosine methyl ester and BTEE.

### Example 6: Synthesis of Microcapsules Using Tyrosine Methyl Ester, BTEE and Papain—Fragrance 3

In a flask, 20 mg papain was dissolved in 2 ml phosphate buffer containing 1 mM EDTA+5 mM Cysteine. The solution was incubated at 40° C. for 10 min.

In another flask equipped with mechanical stirrer and a reflux condenser, 0.26 g Tyrosine methyl ester and 0.1 g BTEE was dispersed in 12 ml 1M phosphate buffer. The substrate solution was pre-incubated at 40° C. for 1 min, followed by addition of the activated enzyme solution to initiate the reaction. The medium was homogenized at 250 rpm for 4 h at 40° C. Then, agitation was increased up to 800 rpm. 3.5 g of fragrance 3 as an oil phase was added into reaction. The aqueous and oil phase were combined and homogenized at 800 rpm at 40° C. The reaction was stopped after 20 h.

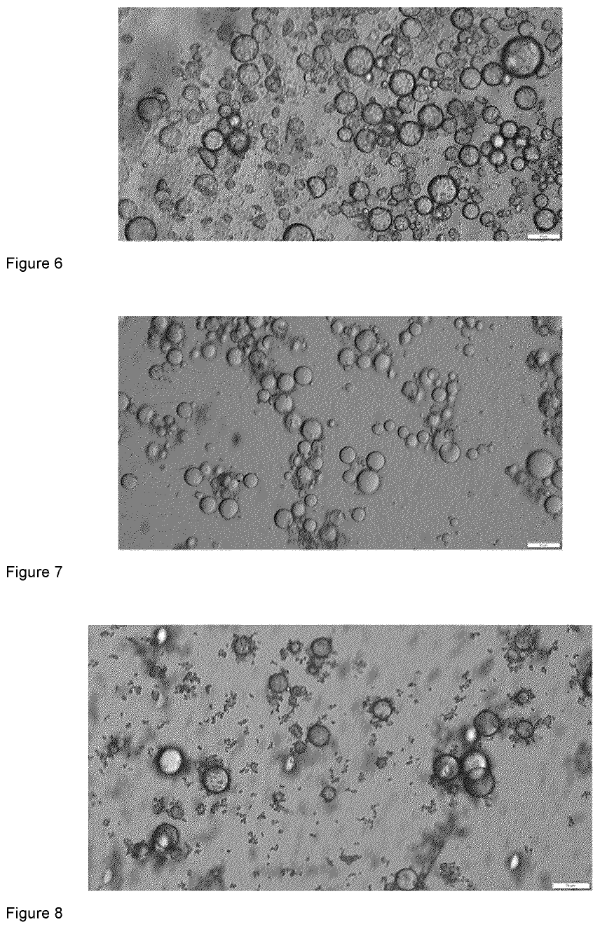

FIG. 6 illustrates a microscopic image of a microcapsule composition encapsulating fragrance 3, obtained by papain-catalysed polymerization of tyrosine methyl ester and BTEE.

### Example 7: Synthesis of Microcapsules Using Tyrosine Methyl Ester, BTEE and Papain—Fragrance 4

In a flask, 20 mg papain was dissolved in 2 ml phosphate buffer containing 1 mM EDTA+5 mM Cysteine. The solution was incubated at 40° C. for 10 min.

In another flask equipped with mechanical stirrer and a reflux condenser, 0.26 g Tyrosine methyl ester and 0.1 g BTEE was dispersed in 12 ml 1M phosphate buffer. The substrate solution was pre-incubated at 40° C. for 1 min, followed by addition of the activated enzyme solution to initiate the reaction. The medium was homogenized at 250 rpm for 4 h at 40° C. Then, agitation was increased up to 800 rpm. 3.5 g of fragrance 4 as an oil phase was added into reaction. The aqueous and oil phase were combined and homogenized at 800 rpm at 40° C. The reaction was stopped after 20 h.

FIG. 7 illustrates a microscopic image of a microcapsule composition encapsulating fragrance 4, obtained by papain-catalysed polymerization of tyrosine methyl ester and BTEE.

### Example 8: Synthesis of Microcapsules Using Tyrosine Methyl Ester, BTEE and Peroxidase Fragrance 2

In a flask, 1.4 mg HRP was dissolved in 2 ml phosphate buffer containing 1 mM EDTA+5 mM Cysteine. The solution was incubated at 40° C. for 10 min.

In another flask equipped with mechanical stirrer and a reflux condenser, 0.26 g Tyrosine methyl ester and 0.1 g BTEE was dispersed in 12 ml 1M phosphate buffer. The substrate solution was pre-incubated at 40° C. for 1 min, followed by addition of the activated enzyme solution to initiate the reaction. The medium was homogenized at 250 rpm for 4 h at 40° C. Then, agitation was increased up to 800 rpm. 3.5 g of fragrance 2 as an oil phase was added into reaction. The aqueous and oil phase were combined and homogenized at 800 rpm at 40° C. for 5 min. 1.5 ml H₂O₂ was added to the mixture drop by drop over 2 hours. The reaction was stopped after 1 h.

FIG. 8 illustrates a microscopic image of a microcapsule composition encapsulating fragrance 2, obtained by peroxidise-catalysed polymerization of tyrosine methyl ester and BTEE.

### Example 9: Synthesis of microcapsules using ω-Pentadecanolide (poly(PDL)) and lipase-Fragrance 1 (18.75% ω-Pentadecanolide)

In a flask, 5 g of ω-Pentadecanolide and 6 g fragrance 1 were stirred at 45° C. to obtain homogenous solution. This solution was added to 40 g of a 1% Pectin A104 solution. The mixture was stirred vigorously (800 rpm) for 1 h at 45° C. to form a stable emulsion. pH was adjusted to 6.2.

A suspension of 200 mg lipase from Pseudomonas cepacia in 20 g of a 1% Pectin A104 solution was added to the emulsion and stirred at 60° C. for 2 h by maintaining the agitation speed at 800 rpm. Then the temperature was increased from 60° C. to 70° C. and the emulsion was stirred for 24 h.

The median particle size by volume has been measured to be 64.9 μm.

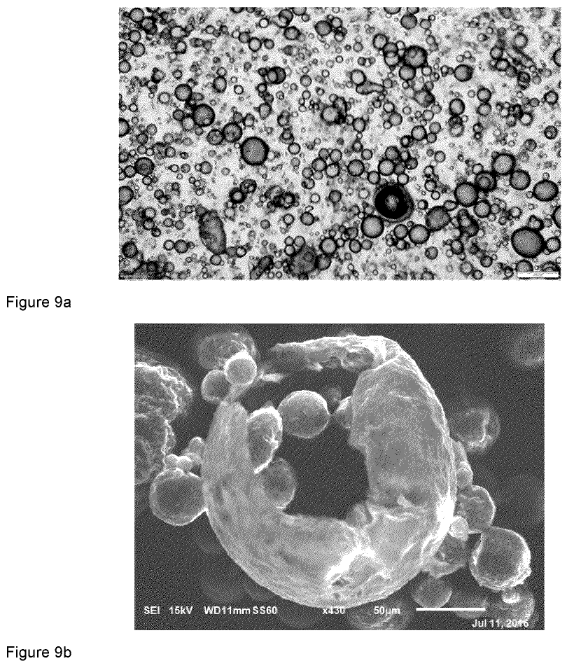

FIG. 9a illustrates a microscopic image of a microcapsule composition encapsulating fragrance 1, obtained by lipase-catalysed polymerization of w-pentadecanolide (at 18.75 wt % monomer concentration).

FIG. 9b shows a SEM (scanning electron microscopy) image of a microcapsule composition encapsulating fragrance 1, obtained by lipase-catalysed polymerization of w-pentadecanolide (at 18.75 wt % monomer concentration).

### Example 10: Synthesis of Microcapsules Using ω-Pentadecanolide (Poly(PDL)) and Lipase—Fragrance 1 (3.75% ω-Pentadecanolide)

In a flask, 1.8 g of ω-Pentadecanolide and 16.2 g fragrance 1 were stirred at 45° C. to obtain homogenous solution. This solution was added to 30 g of a 1% Pectin A104 solution. The mixture was stirred vigorously (800 rpm) for 1 h at 45° C. to form a stable emulsion. pH was adjusted to 5.2.

A suspension of 300 mg lipase from Pseudomonas cepacia in 30 g of a 1% Pectin A104 solution was added to the emulsion and stirred at 60° C. for 7 h by maintaining the agitation speed at 800 rpm.

The median particle size by volume has been measured to be 44 μm.

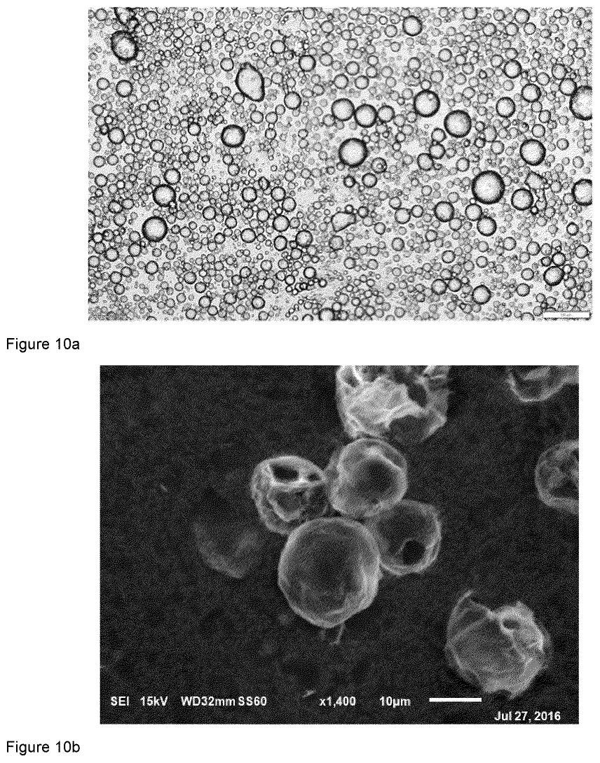

FIG. 10a illustrates a microscopic image of a microcapsule composition encapsulating fragrance 1, obtained by lipase-catalysed polymerization of w-pentadecanolide (at 3.75 wt % monomer concentration).

FIG. 10b shows a SEM (scanning electron microscopy) image of a microcapsule composition encapsulating fragrance 1, obtained by lipase-catalysed polymerization of w-pentadecanolide (at 3.75 wt % monomer concentration).

1. A microcapsule composition comprising at least one core-shell microcapsule, wherein the at least one core-shell microcapsule comprises a hydrophobic core and a shell surrounding the core, wherein the shell comprises a polymer or oligomer generated in situ by an enzyme-catalysed oligomerization/polymerization of a substrate.

2. The microcapsule composition according to claim 1, wherein the core comprises a solvent material, at least one benefit agent, or a mixture thereof.

3. The microcapsule composition according to claim 2, wherein the microcapsule composition comprises a water-soluble matrix in which a benefit agent is entrapped.

4. The microcapsule composition according to claim 1, wherein at least one of the shell surrounding the core and the water-soluble matrix in which a benefit agent is entrapped is bio-based.

5. The microcapsule composition according to claim 1, wherein at least one of the shell surrounding the core, the benefit agent and the water-soluble matrix in which a benefit agent is entrapped is biodegradable.

6. The microcapsule composition according to claim 1, wherein the enzyme is
a) a hydrolase (EC3); or
b) an oxidoreductase (EC1).

7. The microcapsule composition according to claim 1, wherein
a1) the esterase is a carboxylic-ester hydrolase (EC3.1.1);
a2) the protease/peptidase is a serine endopeptidase (EC3.4.21) or a cysteine endopeptidase (EC3.4.22) such as papain;
b) the oxidoreductase is a peroxidase (EC1.11.1).

8. The microcapsule composition according to claim 1, wherein the enzyme is immobilized on a support by covalent binding and/or physical adsorption.

9. The microcapsule composition according to claim 1, wherein the substrate is selected from the group consisting of lactones, cyclic carbonates, polycarboxylic compounds, polyols, cyclic amides/lactams, @-amino acids and their esters, amino acid alkyl esters and their derivatives, oligopeptide alkyl esters, phenyl containing peptides, phenyl containing amino acids or combinations thereof.

10. The microcapsule composition according to claim 1, wherein
a1) when the enzyme is a lipase, the substrate is selected from the group consisting of lactones, cyclic carbonates, polycarboxylic compounds, polyols, cyclic amides/lactams, ω-amino acids and their esters, amino acid alkyl esters and their derivatives or combinations thereof;
a2) when the enzyme is a protease, the substrate is selected from the group consisting of amino acid alkyl esters and their derivatives, oligopeptide alkyl esters;
b) when the enzyme is an oxidoreductase, the substrate is a phenyl-containing amino acid or amino-acid derivative or a phenyl-containing peptide or combinations thereof.

11. The microcapsule composition according to claim 10, wherein
a) the lactones are macrocyclic lactones having more than 12-membered rings;
b) the cyclic carbonates are cyclic carbonates having more than 8-membered rings;
c) the cyclic amides/lactams are lactams having more than 8-membered rings;
d) the polycarboxylic compounds are polycarboxylic compounds having more than 8 carbon atoms;
e) the polyols are polyols having more than 8 carbon atoms;
f) the amino acid alkyl esters and their derivatives are hydrophobic amino acid alkyl esters;
g) The oligopeptide alkyl esters are oligopeptide alkyl esters wherein the peptide comprises 2 to 9 amino acids;
h) the phenyl-containing amino acid is tyrosine or phenylalanine;
i) the phenyl-containing peptide is phenyl-containing peptide wherein the peptide comprises 2 to 9 amino acids;
optionally wherein the alkyl is selected from the group consisting of methyl, ethyl, n-propyl, i-propyl, n-butyl, i-butyl, t-butyl and benzyl.

12. A method of preparing the microcapsule composition according to claim 1, the method comprising the steps of:
a) providing a substrate and an enzyme in an aqueous phase in conditions suitable for providing enzymatic activity;
b) providing a hydrophobic phase;
c) emulsifying the hydrophobic phase in the aqueous phase to form a microcapsule composition.

13. A method according to claim 12, further comprising a step of spray drying the emulsion resulting from step c).

14. A consumer product comprising a microcapsule composition according to claim 2, optionally wherein the consumer product is a fabric care product, a home care product or a personal care product.

15. A method of enhancing the performance of a benefit agent in a consumer product, comprising the use of the microcapsule composition according to claim 2.

16. The microcapsule composition according to claim 2, wherein the benefit agent is a fragrance ingredient.

17. The microcapsule composition according to claim 6, wherein
a) the hydrolase (EC3) is an esterase (EC3.1) or a protease/peptidase (EC3.4); or
b) the oxidoreductase (EC1) is an oxidoreductase that acts on diphenols as donors (EC1.10) or on a peroxide as acceptor (EC1.11).

18. The consumer product according to claim 14, wherein the consumer product is a fabric care product, a home care product or a personal care product.
